(19) United States
(12) Patent Application Publication
(10) Pub. No.: US 2025/0302501 A1
(43) Pub. Date: Oct. 2, 2025

(54) SYRINGE APPARATUS TO FACILITATE SINGLE-OPERATOR ULTRASOUND GUIDED REGIONAL ANESTHESIA

(71) Applicant: Sohan Parekh, Austin, TX (US)

(72) Inventors: Sohan Parekh, Austin, TX (US); Heather Armstrong, Austin, TX (US); Samuel Bello, Baltimore, MD (US); Destiny Harris, Round Rock, TX (US); Emma Laub, Houston, TX (US); Mikul Misra, Round Rock, TX (US); Sapun Parekh, Austin, TX (US)

(21) Appl. No.: 19/091,921

(22) Filed: Mar. 27, 2025

Related U.S. Application Data
(60) Provisional application No. 63/572,993, filed on Apr. 2, 2024.

(51) Int. Cl. A61B 17/34; A61M 5/145

(52) U.S. Cl. CPC A61B 17/3403; A61M 5/1452; A61B 2017/3413

(57) ABSTRACT

An embodiment facilitates a single-operator technique for ultrasound-guided regional anesthesia (UGRA) including ultrasound transducer manipulation, needle placement, and anesthesia injection. This is accomplished via a syringe with, in an embodiment, an mounting plate that universally attaches the fluid reservoir to an ultrasound transducer. The syringe with mounting plate conforms and adheres to the transducer by a foam adhesive and is secured in place with, for example, elastic bands. In an embodiment, the syringe is designed with a Luer lock tip that can be rotated into one of multiple positions as desired by the user. In an embodiment, a plunger that fits the syringe is designed to be operated using one hand with the positioning of a finger loop at its midpoint as well as a three-loop configuration at the end.

### CROSS-REFERENCE TO RELATED APPLICATION

This application claims priority to U.S. Provisional Patent Application No. 63/572,993 filed on Apr. 2, 2024 and entitled “Syringe Apparatus to Facilitate Single-Operator Ultrasound Guided Regional Anesthesia”, the content of which is hereby incorporated by reference.

### BACKGROUND

Nerves are activated by an influx of sodium ions through sodium ion channels, causing depolarization of the neuron membrane. If the depolarization reaches the threshold value of ˜−50 mV, an action potential occurs, conducting the signal along the nerve, allowing the patient to feel pain. Regional anesthesia aims to prevent this process so no or less pain can be felt by the patient. Anesthesia drugs work by decreasing the neuron's membrane permeability to sodium ions by inactivating the ion channels, thereby “blocking” the nerve. To ensure the nerve is fully blocked, enough sodium ion channels must be inactivated to prevent depolarization and the action potential. Therefore, one must surround the nerve with the anesthesia to prevent the vast majority of ion channels from allowing sodium ions to enter the membrane. During regional anesthesia, the placement of the needle adjacent to the nerve is critical. The needle cannot be within the nerve itself as injecting into the nerve can cause lasting damage. This can be detected using tactile feedback during injection—there is a distinct pressure difference that can be felt by the operator when injecting into the nerve versus soft tissue. Similarly, the needle cannot be placed into a blood vessel to avoid injecting anesthesia systemically rather than locally. This can be detected by aspirating the syringe before injecting. If there is blood in the syringe after aspiration, it indicates the needle is in a blood vessel and should be repositioned.

Ultrasound can be used to more accurately guide the needle and apply the anesthetic around the nerve. Ultrasound-guided regional anesthesia (UGRA) allows the physician to visualize the target nerve structures in the local anatomy, track the needle's position around the nerve, and assess the spread of anesthetic around the nerve during and after injection. UGRA has been shown to improve block success rate, decrease onset time, increase prolongation time, and decrease complications of regional anesthesia. Typically, the physician controls the needle and ultrasound transducer, while a second person controls the injection of the anesthetic. More specifically, current methodology for performing UGRA generally requires a two-person operation: one operator to manipulate the ultrasound transducer and navigate the needle in the patient and another operator to perform the injection of anesthesia.

### BRIEF DESCRIPTION OF THE DRAWINGS

Features and advantages of embodiments of the present invention will become apparent from the appended claims, the following detailed description of one or more example embodiments, and the corresponding figures. Where considered appropriate, reference labels have been repeated among the figures to indicate corresponding or analogous elements.

FIGS. 1A, 1B, 1C provide varying views of an embodiment of the invention.

FIGS. 2A, 2B provide varying views of an embodiment of the invention coupled to a transducer.

FIG. 3 depicts a top view of an embodiment of the invention.

FIG. 4 depicts a top view of an embodiment of the invention.

FIGS. 5A, 5B, 5C provide varying views of an embodiment of the invention including a syringe and mounting plate.

FIGS. 6A, 6B provide an embodiment with finger loops or similar restraint mechanism at the lower end of the mounting plate.

FIGS. 7A, 7B provide an embodiment with a mounting plate that has an adjustable width to fit different size transducers.

FIGS. 8A, 8B provide an embodiment with adjustable members affixed on each side of the mounting plate.

FIGS. 9A, 9B provide an embodiment utilizing a harness designed for a specific transducer.

FIGS. 10A, 10B, 10C, 10D provide an embodiment with a polygonal-shaped fluid reservoir.

### DETAILED DESCRIPTION

Reference will now be made to the drawings wherein like structures may be provided with like suffix reference designations. In order to show the structures of various embodiments more clearly, the drawings included herein are diagrammatic representations of structures. Thus, the actual appearance of the fabricated structures, for example in a photo, may appear different while still incorporating the claimed structures of the illustrated embodiments (e.g., walls may not be exactly orthogonal to one another in actual fabricated devices). Moreover, the drawings may only show the structures useful to understand the illustrated embodiments. Additional structures known in the art may not have been included to maintain the clarity of the drawings. For example, not every layer of a device is necessarily shown. “An embodiment”, “various embodiments” and the like indicate embodiment(s) so described may include particular features, structures, or characteristics, but not every embodiment necessarily includes the particular features, structures, or characteristics. Some embodiments may have some, all, or none of the features described for other embodiments. “First”, “second”, “third” and the like describe a common object and indicate different instances of like objects are being referred to. Such adjectives do not imply objects so described must be in a given sequence, either temporally, spatially, in ranking, or in any other manner. “Connected” may indicate elements are in direct physical or electrical contact with each other and “coupled” may indicate elements co-operate or interact with each other, but they may or may not be in direct physical or electrical contact. Phrases such as “comprising at least one of A or B” include situations with A, B, or A and B.

As addressed above, currently two operators are required to perform UGRA procedures: a first operator to position the needle (one hand) while using the ultrasound transducer (other hand) and a second operator to inject the anesthetic when instructed by the first operator. Applicant determined this arrangement reduces the efficiency of the clinical staff and increases the risk of human error as the control of injection is split between two people rather than just one. Therefore, Applicant determined there is a need for a way to administer localized anesthesia by a single operator that maintains the same level of control over both the needle and injection of anesthetic when done by a two-operator team. Further, Applicant determined maintaining tactile feedback and control over the needle, dosing, and injection speed are all desirable and help give the operator more control of the procedure.

An embodiment facilitates a single-operator technique for UGRA including ultrasound transducer manipulation, needle placement, and anesthesia injection. This is accomplished via a syringe with, in an embodiment, an integrated mounting plate that universally attaches the fluid reservoir to an ultrasound transducer. The syringe with mounting plate is, in an embodiment, a single-body unit that conforms and adheres to the transducer by a foam adhesive and is secured in place with, for example, elastic bands. In an embodiment, the syringe is designed with a Luer lock tip that can be rotated into one of multiple positions as desired by the user. In an embodiment, a plunger that fits the syringe is designed to be operated using one hand with the positioning of a finger loop at its midpoint as well as a three-loop configuration at the end.

In an embodiment the mounting plate and syringe are monolithic with each other. As used herein, “monolithic” means two or more pieces are unitary with each other and formed from a single unit without means of traditional coupling techniques, such as mechanical fasteners (e.g., nail, bolt, screw), welds, adhesives, and the like. For example, the mounting plate and syringe may be formed via additive manufacturing, a common mold or extrusion, and the like. However, in other embodiments the mounting plate and syringe are not monolithic with each other.

As used herein, a “Luer” lock is a screw coupling on a syringe that creates a leak-free seal. Care providers can connect needles, tubing, and other equipment to the lock to perform various activities involved in patient care. This fitting is standardized, which permits the interchangeable use of various medical devices. Other embodiments may use alternatives to Luer couplings. Such alternatives may provide locking between tubing/needle and syringe.

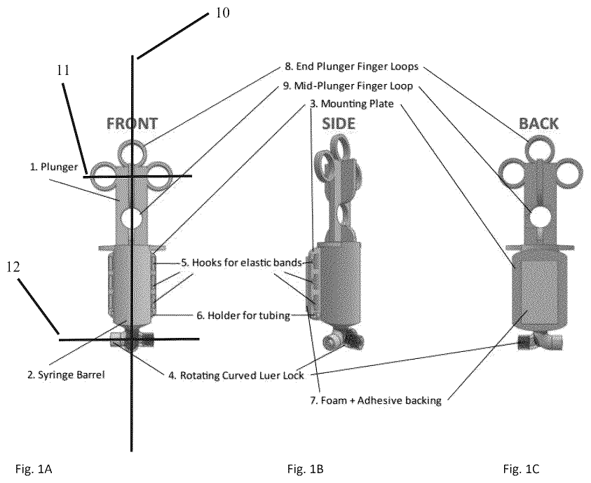

FIGS. 1A, 1B, 1C collectively illustrate an embodiment. The embodiment includes plunger 1 and syringe barrel 2, which includes a cylindrical barrel fluid reservoir terminating in a Luer lock tip. Mounting plate 3 is constructed onto the syringe barrel forming a single body apparatus. Curved Luer lock connector 4 may rotate and lock into one a plurality of positions (e.g., 3 positions). Hooks 5 on the mounting plate allow for elastic bands for securing to transducer. Holder 6 is for tubing carrying anesthetic in order to keep tubing out of the way. Foam and adhesive backing 7 on the mounting plate conforms and adheres to transducer. Three finger loops 8 are located at the end of the plunger. Single finger loop 9 is in the middle of the plunger.

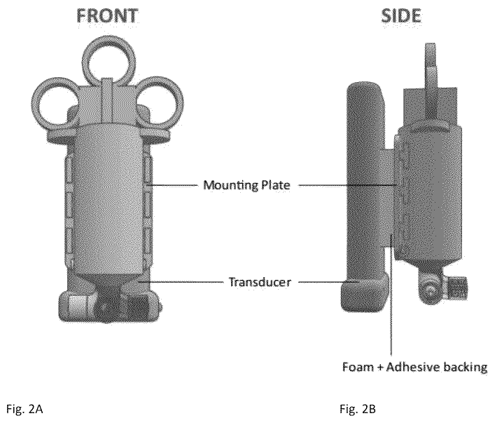

FIGS. 2A, 2B include a syringe apparatus mounted on an ultrasound transducer. The ultrasound transducer is attached to the mounting plate of the syringe apparatus via the foam and adhesive backing addressed with regard to FIGS. 1A, 1B, IC. Elastic bands (not shown) may stretch around the transducer to secure the transducer to the syringe. Mount points for the bands are shown on the mounting plate.

FIG. 3 includes an embodiment with a straight, non-rotating Luer lock tip without curvature or rotation. In an embodiment such a lock tip is substituted for the lock tip of the embodiments of FIGS. 1A and/or 2A.

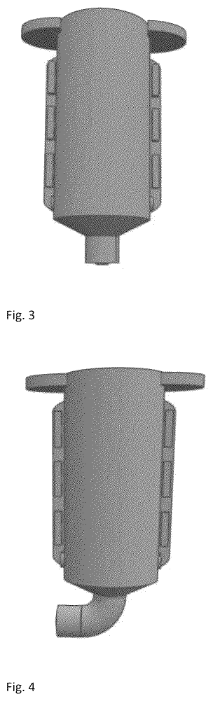

FIG. 4 includes an embodiment with a curved Luer lock tip that is fixed and does not rotate. In an embodiment such a lock tip is substituted for the lock tip of the embodiments of FIGS. 1A and/or 2A.

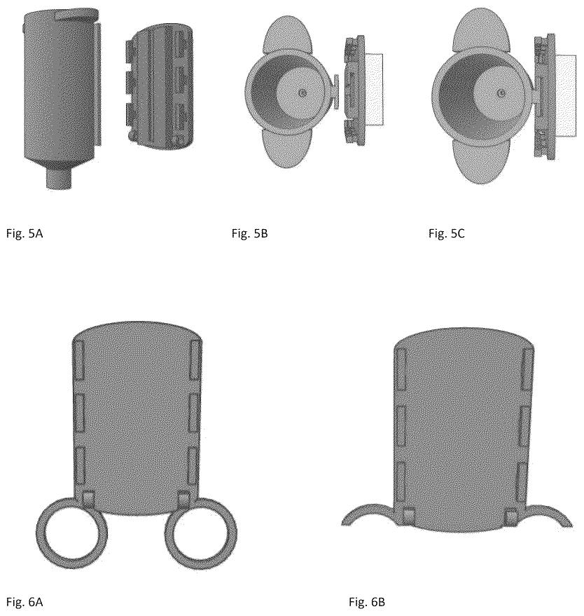

FIGS. 5A, 5B, 5C provide an embodiment with syringe and mounting plate as separate apparatuses which connect via an interlocking channel or other attachment mechanism. Syringe permutations include a rotating Luer lock tip as detailed in FIGS. 1A, 1B, 1C or straight and non-rotating Luer lock tip as addressed above, or a curved Luer lock tip that is fixed and does not rotate as addressed above.

FIGS. 6A, 6B provide embodiments with finger loops or similar restraint mechanism at the lower end of the mounting plate which allow for upward counterforce to be applied by the operator when expelling fluid from the fluid reservoir. FIGS. 6A, 6B are shown without an attached syringe apparatus. As shown in FIG. 1A, the “lower end” of the mounting plate is adjacent the Luer lock and the “upper end” of the mounting plate is adjacent the finger loops of the plunger.

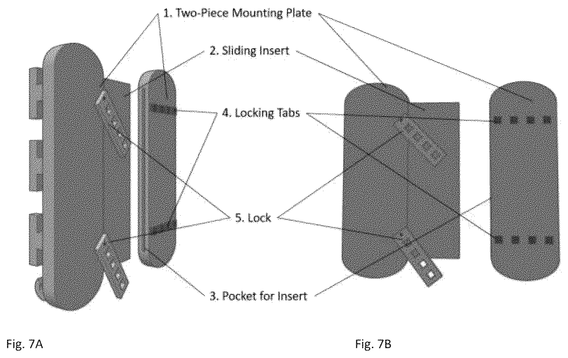

FIGS. 7A, 7B provide an embodiment with a mounting plate that has an adjustable width to fit different size transducers. An embodiment includes a mounting plate divided into multiple pieces (e.g., 2 pieces) where one side has an insert that slides into a pocket on the other side. The position is locked into place by a locking mechanism, such as a perforated elastic band located on the side of the mounting plate that affixes to the transducer. Multiple locking tabs or hooks are spaced at fixed distances to allow various widths thereby creating an adjustable width mounting plate to accommodate multiple transducer widths. In an embodiment, the locking mechanism may be a rigid piece that accommodates any number of locking tabs. Other locking mechanism embodiments are possible as are other implementations to allow for variable widths. FIGS. 7A, 7B are depicted without attached syringe apparatus.

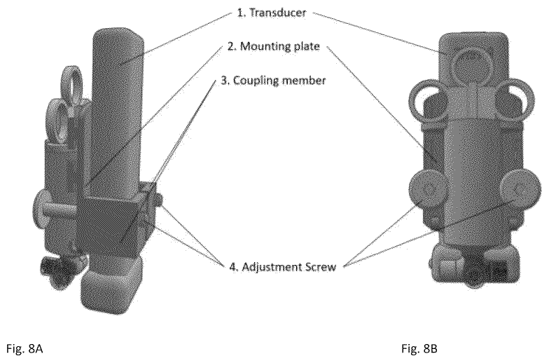

FIGS. 8A, 8B provide an embodiment with adjustable members affixed on each side of the mounting plate. The members allow for universal attachment to the transducer using a mechanism that tightly couples the mounting plate to the transducer. One tightening mechanism includes screws used to adjust the distance to which the coupling members extend from the mounting plate. Other mechanisms for adjusting this distance include a cloth, plastic, or elastic band that traverses through the coupling member and is drawn tightly and secured on the opposite side of the mounting plate from the transducer.

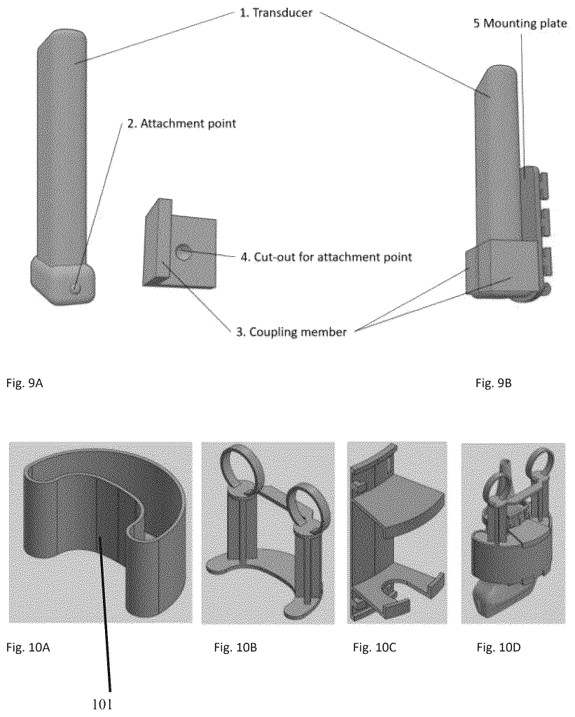

FIGS. 9A, 9B provide an embodiment utilizing a harness designed for a specific transducer utilizing each transducer's “biopsy guide” attachment points (see “attachment point” in FIG. 9A). The harness (see coupling member 3) may be part of a unibody construction with the fluid reservoir (e.g., syringe) or may attach to the fluid reservoir via a channel or similar system as detailed in FIGS. 5A, 5B, 5C. Coupling member 3 may provide enough “give” or flex to flex outwardly to allow sliding the transducer into the coupling member whereby the portion 4 “clicks” onto point 2 when the transducer is fully assembled to the bracket.

FIGS. 10A, 10B, 10C, 10D provide an embodiment with a polygonal-shaped fluid reservoir (e.g., FIGS. 10A, 10B include a C-shaped reservoir and complementary C-shaped plunger), which sits in a harness (FIG. 10C) that is attached to the transducer by adhesive foam and tape and secured with clastic bands. The C-shaped reservoir is designed to fit in the curve of the operator's palm that is formed when gripping the transducer.

An embodiment (not shown) includes a malleable or bendable or flexible or resilient bulb apparatus for the fluid reservoir. The bulb is attached to the mounting plate in a unibody construction or connected to the mounting plate in a manner like that in FIGS. 5A-5C. The operator may squeeze the bulb to expel fluid out of the bulb. The bulb may terminate in a Luer lock which attaches to tubing that conveys fluid for injection. The Luer lock may take the form of a curved, rotating design as detailed in FIG. 1A, a straight and non-rotating Luer lock tip at addressed above, or a curved Luer lock tip that is fixed and does not rotate as addressed above.

An embodiment includes an accordion style cylindrical syringe fluid reservoir which allows for a reduced force necessary to compress the plunger and expel fluid. The syringe may terminate in a Luer lock that attaches to tubing that conveys fluid for injection. The Luer lock may take the form of a curved, rotating design as detailed in FIG. 1A, a straight and non-rotating Luer lock tip as addressed above, or a curved Luer lock tip that is fixed and does not rotate as addressed above.

An embodiment, such as any of those described herein, may be constructed with materials that can be sterilized using a conventional process such as autoclaving and then reused following this sterilization process.

Any of the embodiments described herein may facilitate the performance of UGRA by a single operator. In an embodiment of a process for using systems described herein, following the initial set up for the procedure, the operator begins by attaching the bracket to the ultrasound transducer. Next, the operator will fill the syringe with anesthetic and attach tubing and a needle to the syringe. The operator will hold the transducer/syringe combination system in his or her non-dominant hand and the needle in his or her dominant hand. The user's index finger of his or her non-dominant hand will be placed on the plunger of the syringe so the user can control the injection as needed. The operator moves the needle and ultrasound transducer as needed until the user finds the desired nerve and injects the anesthetic around it. In embodiments where the syringe is separable from the rest of the system, when the procedure is over the syringe can be disposed and the rest of the system can be sterilized to be used again. The order of the above steps may vary in different embodiments (e.g., operator may reverse these steps by filling the fluid and attaching the needle/tubing prior to mounting the apparatus on the transducer).

Note finger placement at the midpoint or end of the plunger is not necessary (nor prohibitive) for maneuvering the system. However, in some embodiments the finger is placed in those locations to allow for depression of the plunger and injection of fluid.

Various example sets are now addressed. A second example may be included in a second example set. If the second example refers to a first example, the second example is referring to a first example in the second example set (not the first example set).

### First Example Set

1. A system comprising: a syringe barrel; a plunger included in the syringe barrel; a Luer lock coupled to a distal end of the syringe barrel; a plate directly connected to the syringe barrel; and an ultrasound transducer releasably coupled to the plate.

As used herein, “releasably coupled” connotes an ability to couple a first object to a second object and the, afterwards, release the first object from the second object.

As used herein, a “transducer” may include an ultrasound wand, and the like. In various embodiments, another portion of imaging equipment may be releasably coupled to the plate.
2. The system of example 1, wherein: the plunger includes first, second, and third apertures at a proximal end of the plunger; the plunger includes a long axis that intersects the second aperture but not the first and third apertures.

See, for example, axis 10 in FIG. 1A.
3. The system of example 2, wherein the plunger includes a fourth aperture and the long axis of the plunger intersects the fourth aperture.
4. The system according to any of examples 2-3, wherein another axis, which is orthogonal to the long axis of the plunger, intersects the first and third apertures but not the second aperture. See, for example, axis 11 of FIG. 1A.
5. The system according to any of examples 1-4, wherein: the plunger includes a long axis; the Luer lock includes a channel having a proximal input and a distal output; the channel is to communicate a fluid from the syringe barrel to the proximal input and then trough the channel and then out the distal output; the Luer lock is permanently coupled to the syringe barrel; the Luer lock is pivotable at least 45 degrees within a plane that is orthogonal to the long axis of the plunger.

See, for example, plane 12 in FIG. 1A. In various embodiments, the Luer lock is pivotable at least 90, 135, 180, 225, 270 degrees or more within the plane that is orthogonal to the long axis of the plunger.
6. The system of example 5, wherein the Luer lock channel is curved in a plane that is parallel to the long axis of the plunger.

For example, in FIG. 1C the Luer lock input is generally vertical but the Luer lock output is generally horizontal.
7. The system of example 6, wherein the Luer lock channel is curved with degree or curvature that is between 80 and 100 degrees.

As used herein, a degree of curvature is defined as the central angle to the ends of an agreed length of either an arc or a chord. In FIG. 1C, the channel has a degree of curvature that equals 90 degrees.
8. The system according to any of examples 1-7 comprising at least one resilient band, wherein the at least one resilient band forces the ultrasound transducer against the plate.
9. The system according to any of examples 1-7, wherein the syringe barrel is keyed to the plate.

For example, see FIG. 5C.
10. The system according to example 9, wherein the syringe barrel includes one of a male or female connector and the plate includes another of the male or female connector that is keyed to the connector of the syringe barrel.
11. The system according to any of examples 1-10 including at least one aperture at a distal end of at least one of the plunger, the plate, or combinations thereof.

For example, see FIG. 6A.
12. The system according to any of examples 1-11, wherein the ultrasound transducer is resiliently coupled to the plate.

For example, resilient bands may couple the transducer to the plate.
13. The system according to any of examples 1-11, wherein the ultrasound transducer is non-resiliently coupled to the plate.

See, for example, FIGS. 8A-8B.
14. The system of any of examples 12-13, wherein: the plate includes at least one sub-plate; the at least one sub-plate is keyed to the ultrasound transducer to releasably coupled the ultrasound transducer to the at least one sub-plate.

See, for example, FIGS. 9A-9B.
15. The system of any of examples 1-14, wherein the syringe barrel includes a non-circular cross-section taken orthogonally to a long axis of the syringe barrel.

See, for example, FIG. 10A.
16. The system of example 15, wherein: an axis is orthogonal to the long axis of the syringe barrel; the axis intersects the ultrasound transducer and first and second opposing lateral sections of the syringe barrel.

For example, such a design may form void 101 (FIG. 10A) to receive the ultrasound transducer. Such a device allows an operator to hold and use the syringe and transducer in one hand while keeping the needle alone in the other hand. In an embodiment, a C-shaped syringe may conform to the shape of a linear transducer and hold over 35 mL of fluid. The plunger may include 2 pillars that are offset from the center. See, for example, FIG. 10B. This offset places the pillar closer to the user's index finger's natural resting position while holding an ultrasound transducer, allowing the user to have smoother control over the plunger's movement. There are 2 pillars on the plunger to accommodate right- and left-handed users. The syringe may attach to the transducer via an attachment piece made up of a plastic bracket that the syringe may snap into. See, for example, FIG. 10C. An embodiment may include rubber straps to bracket device to the transducer.

### Second Example Set

1. A system comprising: a syringe barrel; a plunger included in the syringe barrel; a Luer lock coupled to a distal end of the syringe barrel; a plate directly connected to the syringe barrel and configured to releasably couple to an ultrasound transducer.
2. The system of example 1, wherein: the plunger includes first, second, and third apertures at a proximal end of the plunger; the plunger includes a long axis that intersects the second aperture but not the first and third apertures.
3. The system of example 2, wherein the plunger includes a fourth aperture and the long axis of the plunger intersects the fourth aperture.
4. The system according to any of examples 2-3, wherein another axis, which is orthogonal to the long axis of the plunger, intersects the first and third apertures but not the second aperture.
5. The system according to any of examples 1-4, wherein: the plunger includes a long axis; the Luer lock includes a channel having a proximal input and a distal output; the channel is to communicate a fluid from the syringe barrel to the proximal input and then trough the channel and then out the distal output; the Luer lock is permanently coupled to the syringe barrel; the Luer lock is pivotable at least 45 degrees within a plane that is orthogonal to the long axis of the plunger.
6. The system of example 5, wherein the Luer lock channel is curved in a plane that is parallel to the long axis of the plunger.
7. The system of example 6, wherein the Luer lock channel is curved with degree or curvature that is between 80 and 100 degrees.
8. The system according to any of examples 1-7 comprising at least one resilient band, wherein the at least one resilient band is configured to force the ultrasound transducer against the plate.
9. The system according to any of examples 1-7, wherein the syringe barrel is keyed to the plate.
10. The system according to example 9, wherein the syringe barrel includes one of a male or female connector and the plate includes another of the male or female connector that is keyed to the connector of the syringe barrel.
11. The system according to any of examples 1-10 including at least one aperture at a distal end of at least one of the plunger, the plate, or combinations thereof.
12. The system according to any of examples 1-11, wherein the ultrasound transducer is resiliently coupled to the plate when the system is fully assembled.
13. The system according to any of examples 1-11, wherein the ultrasound transducer is non-resiliently coupled to the plate when the system is fully assembled.
14. The system of any of examples 12-13, wherein: the plate includes at least one sub-plate; the at least one sub-plate is keyed to the ultrasound transducer to releasably couple the ultrasound transducer to the at least one sub-plate.
15. The system of any of examples 1-14, wherein the syringe barrel includes a non-circular cross-section taken orthogonally to a long axis of the syringe barrel.
16. The system of example 15, wherein: an axis is orthogonal to the long axis of the syringe barrel; the axis intersects the ultrasound transducer and first and second opposing lateral sections of the syringe barrel when the system is fully assembled.

### Third Example Set

1. A system comprising: a syringe barrel; a plunger to be included in the syringe barrel; a lock coupled to a distal end of the syringe barrel, the lock being configured to couple to a conduit; a bracket directly connected to the syringe barrel and configured to releasably couple to a medical device.

The conduit may include, for example, a needle or tube.
2. The system of example 1, wherein: the plunger includes first, second, and third apertures at a proximal end of the plunger; the plunger includes a long axis that intersects the second aperture but not the first and third apertures.
3. The system of example 2, wherein the plunger includes a fourth aperture and the long axis of the plunger intersects the fourth aperture.
4. The system according to any of examples 2-3, wherein another axis, which is orthogonal to the long axis of the plunger, intersects the first and third apertures but not the second aperture.
5. The system according to any of examples 1-4, wherein: the plunger includes a long axis; the lock includes a channel having a proximal input and a distal output; the channel is to communicate a fluid from the syringe barrel to the proximal input and then trough the channel and then out the distal output; the lock is permanently coupled to the syringe barrel; the lock is pivotable at least 45 degrees within a plane that is orthogonal to the long axis of the plunger.
6. The system of example 5, wherein the lock channel is curved in a plane that is parallel to the long axis of the plunger.
7. The system of example 6, wherein the lock channel is curved with degree or curvature that is between 80 and 100 degrees.
8. The system according to any of examples 1-7 comprising at least one resilient band, wherein the at least one resilient band is configured to force the ultrasound medical device against the plate.
9. The system according to any of examples 1-7, wherein the syringe barrel is keyed to the plate.
10. The system according to example 9, wherein the syringe barrel includes one of a male or female connector and the bracket includes another of the male or female connector that is keyed to the connector of the syringe barrel.
11. The system according to any of examples 1-10 including at least one aperture at a distal end of at least one of the plunger, the bracket, or combinations thereof.
12. The system according to any of examples 1-11, wherein the ultrasound transducer is resiliently coupled to the bracket when the system is fully assembled.
13. The system according to any of examples 1-11, wherein the medical device is non-resiliently coupled to the bracket when the system is fully assembled.
14. The system of any of examples 12-13, wherein: the bracket includes at least one sub-bracket; the at least one sub-bracket is keyed to the medical device to releasably couple the medical device to the at least one sub-bracket.
15. The system of any of examples 1-14, wherein the syringe barrel includes a non-circular cross-section taken orthogonally to a long axis of the syringe barrel.
16. The system of example 15, wherein: an axis is orthogonal to the long axis of the syringe barrel; the axis intersects the medical device and first and second opposing lateral sections of the syringe barrel when the system is fully assembled.

### Fourth Example Set

1. A system comprising: a fluid reservoir; an applicator coupled to the fluid reservoir; an output conduit coupled to a distal end of the fluid reservoir; and a plate coupled to the fluid reservoir and configured to releasably couple to an imaging transducer.

The applicator may include, for example, a plunger, piston, lever, squeezable bulb, ball, rod, valve, port, and the like.

The plate may be planar, curved, malleable, flexible, stiff, plastic, metal, silicone, and the like.

Alternative version of 1. A system comprising: a fluid reservoir; an applicator coupled to the fluid reservoir; an output conduit coupled to a distal end of the fluid reservoir; and a plate coupled to the fluid reservoir and configured to releasably couple to an imaging wand.
2. The system of example 1, wherein: the applicator includes first, second, and third apertures at a proximal end of the applicator; the applicator includes a long axis that intersects the second aperture but not the first and third apertures.

Alternative version of 2. The system of example 1, wherein: the applicator includes a plurality of apertures at a proximal end of the applicator; the applicator includes a long axis that intersects one of the plurality of apertures but not another of the plurality of apertures.
3. The system of example 2, wherein the applicator includes a fourth aperture and the long axis of the applicator intersects the fourth aperture.
4. The system according to any of examples 2-3, wherein another axis, which is orthogonal to the long axis of the applicator, intersects the first and third apertures but not the second aperture.
5. The system according to any of examples 1-4, wherein: the applicator includes a long axis; the output conduit includes a channel having a proximal input and a distal output; the channel is to communicate fluid from the fluid reservoir to a recipient via the proximal input, and then the channel, and then the distal output; the output conduit is coupled to the fluid reservoir; the output conduit is pivotable at least 45 degrees within a plane that is orthogonal to the long axis of the applicator.

The recipient may include a patient, conduit, and the like.

Another version of 5. The system according to any of examples 1-4, wherein: the applicator includes a long axis; the output conduit includes a channel having a proximal input and a distal output; the channel is to communicate fluid from the fluid reservoir to a recipient via the proximal input, and then the channel, and then the distal output; the output conduit is coupled to the fluid reservoir.
6. The system of example 5, wherein the output conduit channel is curved in a plane that is parallel to the long axis of the applicator.
7. The system of example 6, wherein the output conduit channel is curved with degree or curvature that is between 80 and 100 degrees.
8. The system according to any of examples 1-7 comprising at least one resilient band, wherein the at least one resilient band is configured to force the imaging transducer against the plate.
9. The system according to any of examples 1-7, wherein the fluid reservoir is keyed to the plate.
10. The system according to example 9, wherein the fluid reservoir includes one of a male or female connector and the plate includes another of the male or female connector that is keyed to the connector of the fluid reservoir.
11. The system according to any of examples 1-10 including at least one aperture at a distal end of at least one of the applicator, the plate, or combinations thereof.
12. The system according to any of examples 1-11, wherein the imaging transducer is resiliently coupled to the plate when the system is fully assembled.
13. The system according to any of examples 1-11, wherein the imaging transducer is non-resiliently coupled to the plate when the system is fully assembled.
14. The system of any of examples 12-13, wherein: the plate includes at least one sub-plate; the at least one sub-plate is keyed to the imaging transducer to releasably couple the imaging transducer to the at least one sub-plate.
15. The system of any of examples 1-14, wherein the fluid reservoir includes a non-circular cross-section taken orthogonally to a long axis of the fluid reservoir.
16. The system of example 15, wherein: an axis is orthogonal to the long axis of the fluid reservoir; the axis intersects the imaging transducer and first and second opposing lateral sections of the fluid reservoir when the system is fully assembled.

FIFTH EXAMPLE SET
1. A method for performing imaging-guided regional anesthesia comprising: obtaining a system including: (1) a fluid reservoir; (2) an applicator coupled to the fluid reservoir; (3) an output conduit coupled to a distal end of the fluid reservoir; and (4) a plate coupled to the fluid reservoir and configured to releasably couple to an imaging transducer; coupling the plate to the transducer; coupling a conduit and needle to the output conduit; holding the transducer and system in a first hand of the operator and the needle in a second hand of the operator; placing a finger of the first hand on the applicator and moving the transducer to image a portion of a patient; moving the needle with the second hand until the needle is adjacent a nerve; operating the applicator with the finger of the first hand to deploy a fluid from the fluid reservoir into the patient while holding the needle with the second hand.
2. The method of example 1, wherein the fluid includes an anesthetic.
3. The method of example 2, wherein: the applicator includes a plunger; operating the applicator with the finger of the first hand to deploy a fluid from the fluid reservoir into the patient includes advancing the plunger within the fluid reservoir.

The foregoing description of the embodiments of the invention has been presented for the purposes of illustration and description. It is not intended to be exhaustive or to limit the invention to the precise forms disclosed. This description and the claims following include terms, such as left, right, top, bottom, over, under, upper, lower, first, second, etc. that are used for descriptive purposes only and are not to be construed as limiting. For example, terms designating relative vertical position refer to a situation where a side of a substrate is the “top” surface of that substrate; the substrate may actually be in any orientation so that a “top” side of a substrate may be lower than the “bottom” side in a standard terrestrial frame of reference and still fall within the meaning of the term “top.” The term “on” as used herein (including in the claims) does not indicate that a first layer “on” a second layer is directly on and in immediate contact with the second layer unless such is specifically stated; there may be a third layer or other structure between the first layer and the second layer on the first layer. The embodiments of a device or article described herein can be manufactured, used, or shipped in a number of positions and orientations. Persons skilled in the relevant art can appreciate that many modifications and variations are possible in light of the above teaching. Persons skilled in the art will recognize various equivalent combinations and substitutions for various components shown in the Figures. It is therefore intended that the scope of the invention be limited not by this detailed description, but rather by the claims appended hereto.

What is claimed is:

1. A system comprising:
a syringe barrel;
a plunger included in the syringe barrel;
a Luer lock coupled to a distal end of the syringe barrel;
a plate directly connected to the syringe barrel; and
an ultrasound transducer releasably coupled to the plate.

2. The system of claim 1, wherein:
the plunger includes first, second, and third apertures at a proximal end of the plunger;
the plunger includes a long axis that intersects the second aperture but not the first and third apertures.

3. The system of claim 2, wherein the plunger includes a fourth aperture and the long axis of the plunger intersects the fourth aperture.

4. The system of claim 2, wherein another axis, which is orthogonal to the long axis of the plunger, intersects the first and third apertures but not the second aperture.

5. The system of claim 4, wherein:
the plunger includes a long axis;
the Luer lock includes a channel having a proximal input and a distal output;
the channel is to communicate a fluid from the syringe barrel to the proximal input and then trough the channel and then out the distal output;
the Luer lock is permanently coupled to the syringe barrel;
the Luer lock is pivotable at least 45 degrees within a plane that is orthogonal to the long axis of the plunger.

6. The system of claim 5, wherein the channel is curved in a plane that is parallel to the long axis of the plunger.

7. The system of claim 6, wherein the channel is curved with a degree of curvature that is between 80 and 100 degrees.

8. The system of claim 1 comprising at least one resilient band, wherein the at least one resilient band is to force the ultrasound transducer against the plate.

9. The system of claim 1, wherein the syringe barrel is keyed to the plate.

10. The system according to claim 9, wherein the syringe barrel includes one of a male or female connector and the plate includes another of the male or female connector that is keyed to the one of a male or female connector of the syringe barrel.

11. The system of claim 1 including at least one aperture at a distal end of at least one of the plunger, the plate, or combinations thereof.

12. The system of claim 1, wherein the ultrasound transducer is resiliently coupled to the plate.

13. The system of claim 12, wherein:
the plate includes at least one sub-plate;
the at least one sub-plate is keyed to the ultrasound transducer to releasably coupled the ultrasound transducer to the at least one sub-plate.

14. The system of claim 1, wherein the ultrasound transducer is non-resiliently coupled to the plate.

15. The system of claim 1, wherein the syringe barrel includes a non-circular cross-section taken orthogonally to a long axis of the syringe barrel.

16. The system of claim 15, wherein:
an axis is orthogonal to the long axis of the syringe barrel;
the axis intersects the ultrasound transducer and first and second opposing lateral sections of the syringe barrel.

17. A system comprising:
a syringe barrel;
a plunger included in the syringe barrel;
a Luer lock coupled to a distal end of the syringe barrel;
a plate directly connected to the syringe barrel and configured to releasably couple to an ultrasound transducer.

18. The system of claim 17, wherein:
the plunger includes first, second, and third apertures at a proximal end of the plunger;
the plunger includes a long axis that intersects the second aperture but not the first and third apertures.

19. The system of claim 18, wherein another axis, which is orthogonal to the long axis of the plunger, intersects the first and third apertures but not the second aperture.

20. The system of claim 16, wherein:
the plunger includes a long axis;
the Luer lock includes a channel having a proximal input and a distal output;
the channel is to communicate a fluid from the syringe barrel to the proximal input and then trough the channel and then out the distal output;
the Luer lock is permanently coupled to the syringe barrel;
the Luer lock is pivotable at least 45 degrees within a plane that is orthogonal to the long axis of the plunger.
